(19) United States
(12) Patent Application Publication
(10) Pub. No.: US 2017/0138924 A1
(43) Pub. Date: May 18, 2017

(54) AUTOMATED CELL CULTURE SYSTEM AND CORRESPONDING METHODS

(71) Applicants: ROSS BEIGHLEY, NORTHBOROUGH, MA (US); CONNECTICUT CHILDREN'S MEDICAL CENTER, HARTFORD, CT (US)

(72) Inventors: Ross BEIGHLEY, NORTHBOROUGH, MA (US); Min TANG-SCHOMER, MARLBOROUGH, CT (US)

(21) Appl. No.: 15/320,002

(22) PCT Filed: Jun. 19, 2015
(86) PCT No.: PCT/US2015/036708

§ 371 Date: Dec. 19, 2016

Related U.S. Application Data
(60) Provisional application No. 62/015,025, filed on Jun. 20, 2014.

(51) Int. Cl. G01N 33/483; G06F 19/00; G06F 19/18; G01N 21/77

(52) U.S. Cl. CPC G01N 33/4833; G01N 21/77; G06F 19/345; G06F 19/18

(57) ABSTRACT

Described herein are systems, methods and computer program products for real time assessment and monitoring of biological cells or their environment. In the systems and methods, a computer receives readouts of the biological cells or their environment and assesses changes in the biological cells or their environment by comparing current and previous values of parameters that describe the biological cells or their environment. The computer then determines treatments to be applies to the cells based on the assessing and user-specified criteria and then applies the treatment by controlling a setting on an environmental control unit to cause the treatment to be applied to the biological cells. The systems and methods described herein provide an automated system that includes computer-driven modifications that eliminate the need for the researcher to be present and controlling the flow of the experiment as it is being run.

### FIELD OF THE DISCLOSURE

The present disclosure is related to a laboratory automation feedback system for allowing remote and/or automatic monitoring, control and interaction with ongoing laboratory experiments, and more particularly but not exclusively, to a laboratory automation feedback system having at least one imaging device and a computer for capturing and analyzing images to provide ongoing experimental results and automatic result-based modifications to ongoing protocols while also allowing remote users to monitor the experiment, interact with, control and communicate changes to the experiment in real-time.

### BACKGROUND

Laboratory facilities that provide controlled conditions where scientific experiments are performed, for example on specimens, in order to obtain information needed for research and development or for diagnostic purposes are well known in the art. Scientists and lab workers use a variety of laboratory equipment to perform testing, or experiments, which vary depending upon the particular field of study. One common piece of lab equipment is the microscope, which is used to provide a magnified image of a specimen or object in order that the specimen may be seen clearly. A variety of different types of microscopes may be utilized in a laboratory, for example light microscopes, electron microscopes and scanning probe microscopes. The most recent developments in light microscopes largely center on the rise of fluorescence microscopy in biology and fluorescent labeling of cellular structures.

Fluorescent labeling uses different fluorophores for analysis of biological cell structure at a molecular level in both live and fixed samples, for example by chemical staining of cellular structures. Such experiments can be time consuming as cell fluorescence is measured at timed intervals and drug responses are administered in response to the collected data. Fluorescence experiments like many conventional biological, chemical, and pharmaceutical experiments and procedures are manually intensive and have not changed much over the years. These experiments require that a human operator manually conduct most, if not every aspect of the experiment, and respond to data manually, which is a tedious time consuming and repetitive process that is prone to error.

In one conventional fluorescent experiment, a biology researcher manually plates fluorescently tagged cells on a slide and views them under a fluorescent microscope capable of measuring the minute quantities of light generated by the cells. The researcher administers chemicals to the cells and records changes to the intensity of the fluorescence measured by the microscope in order to maintain a specific static or dynamic environment. If the intensity falls below or above a chosen threshold the researcher determines whether or not to administer a new dose of chemical and the process continues. After sufficient data has been collected, the fluorescence of the cells deteriorates, or the cells' exposure to environmental conditions reaches a maximum, the researcher ends the experiment and the accumulated data is then analyzed. Ideally, a new pattern involving cell function or homeostatic mechanism under certain environmental stimulus can be discerned. Conventionally this fluorescence experiment requires approximately two hours of an experienced lab worker's undivided attention, which leaves large room for error in the data collected. Because the experiment is so labor-intensive many other experiments are performed that require less effort, but yield less data.

Laboratory automation that allows for remote interaction with laboratory experiments has been developed in various fields with varying success. Automated laboratory testing systems have not generally been implemented due to a number of reasons including the cost of specialized equipment, reliability, and ability of the remote access the experiment and results in the same way as a human lab-worker in a cost-effective manner.

One type of remote diagnostic system is disclosed in U.S. Pub. No. US 2002/0106119 entitled Collaborative Diagnostic System. The diagnostic system includes a microscope to provide an image of a biological specimen in digital form for comparison to a database of pathologies in order to identify the pathologies that are candidates for the pathologies associated with the specimen. The application also discloses the use of a decision support system that processes the image from the microscope to obtain an image profile which is compared to the image profile of the pathologies in the database, a diagnostic system including a computer assisted evaluation of objective characteristics of pathologies and a client interface for allowing a remote user to receive the image of the biological specimen, send a signal to control the microscope to adjust the image, and to communicate with other remote users. The diagnostic system disclosed allows for collaboration between remote users in order to discriminate among pathologies with similar characteristics and remote control of the image-taking microscope to aid in collaboration. However, the database of pathologies requires a large investment and there is still the need for human workers to actually perform the visual comparison in certain cases, thus creating a gap in the experimental feedback system.

### BRIEF SUMMARY

In one aspect, a method for real-time assessment and monitoring of biological cells or their environment comprises

receiving a current readout of the biological cells or their environment;

assessing, by a computer, a change in the biological cells or their environment by comparing a current value of a parameter that describes the biological cells or their environment to a previous value of the parameter for the biological cells or their environment and/or a current or previous value for a comparative sample, wherein the current value of the parameter that describes the biological cells or their environment is determined from the current readout;

determining, by the computer, a treatment to be applied to the biological cells or their environment, the determining based on the assessing and on user specified criteria;

applying, by the computer, the treatment to the biological cells or their environment, the applying comprising controlling a setting on an environmental control unit to cause the treatment to be applied to the biological cells; and

optionally performing the assessing, determining, and applying on a periodic basis in response to receiving additional current readouts of the biological cells or their environment.

In another aspect, a system for real-time assessment and monitoring of biological cells or their environment comprises

a means for interfacing to a microscope,

a cell chamber for obtaining microscopic images of the biological cells or their environment using the microscope, the cell chamber comprising an inlet and an outlet, wherein the inlet and the outlet allow for flow of a reagent through the cell chamber,

a reagent handling unit in fluid communication with the inlet of the cell chamber for dispensing reagent through the inlet of the cell chamber, and

a computer configured for accepting the microscopic images from the microscope, for processing the microscopic images from the microscope, and for controlling the reagent handling unit based on the processing.

In a still further aspect, a system for real-time assessment and monitoring of biological cells or their environment comprises

a memory having computer readable instructions; and

an environmental control unit, and one or more processors for executing the computer readable instructions, the computer readable instructions comprising:

receiving a current readout of the biological cells or their environment;

assessing a change in the biological cells or their environment by comparing a current value of a parameter that describes the biological cells or their environment to a previous value of the parameter for the biological cells or their environment and/or a current or previous value for a comparative sample, wherein the current value of the parameter that describes the biological cells or their environment is determined from the current readout;

determining a treatment to be applied to the biological cells or their environment, the determining based on the assessing and on user specified criteria;

applying the treatment to the biological cells or their environment, the applying comprising controlling a setting on the environmental control unit to cause the treatment to be applied to the biological cells; and

optionally performing the assessing, determining, and applying on a periodic basis in response to receiving additional current readouts of the biological cells or their environment.

In a still further aspect, a computer program product for real-time assessment and monitoring of biological cells or their environment comprises

a computer readable storage medium having program instructions embodied therewith, the program instructions executable by a processor to cause the processor to:

receive a current readout of the biological cells or their environment;

assess a change in the biological cells or their environment by comparing a current value of a parameter that describes the biological cells or their environment to a previous value of the parameter for the biological cells or their environment and/or a current or previous value for a comparative sample, wherein the current value of the parameter that describes the biological cells or their environment is determined from the current readout;

determine a treatment to be applied to the biological cells or their environment, the determining based on the assessing and on user specified criteria;

apply the treatment to the biological cells or their environment, the applying comprising controlling a setting on the environmental control unit to cause the treatment to be applied to the biological cells; and

optionally perform the assessing, determining, and applying on a periodic basis in response to receiving additional current readouts of the biological cells or their environment.

### BRIEF DESCRIPTION OF THE DRAWINGS

FIG. 1 is a flow chart of the laboratory automation system according to one embodiment of the present disclosure.

FIGS. 2a-2d are screen shots of an embodiment of a user interface of the laboratory automation system of FIG. 1.

FIG. 3 is a schematic of an embodiment of the conceptual design of a system of the present disclosure for personalized drug development.

FIG. 4 is a schematic of an embodiment of a prototype system of the present disclosure. The software is an iPhone app for remote monitoring.

FIG. 5 is a schematic of designs for an experimental chamber in accordance with embodiments.

FIG. 6 is a schematic of a workflow for cancer drug screening in accordance with an embodiment.

FIG. 7 is a block diagram of a laboratory automation system in accordance with an embodiment.

Various aspects of at least one embodiment are discussed below with reference to the accompanying figures, which are not necessarily drawn to scale, emphasis instead being placed upon illustrating the principles disclosed herein. The figures are included to provide an illustration and a further understanding of the various aspects and embodiments, and are incorporated in and constitute a part of this specification, but are not intended as a definition of the limits of any particular embodiment. The figures, together with the remainder of the specification, serve to explain principles and operations of the described and claimed aspects and embodiments. In the figures, each identical or nearly identical component that is illustrated in various figures is represented by a like numeral. For purposes of clarity, not every component may be labeled in every figure.

### DETAILED DESCRIPTION

Many systems, such as the collaborative diagnostic system described in the U.S. Pub. No. US 2002/0106119 do not allow for real-time intervention with an ongoing experiment in order to allow the parameters of the experiment to be changed, as may be needed, while the experiment is being run. Moreover, the current systems, when they do allow intervention, require user intervention and do not place the capability in the hands of ‘intelligent’ computer driven modules and routines that are programmed to make decisions based on programmed parameters. Current systems do not allow for real time intervention with a sample because of the time lag between the user deciding that a change should be made, e.g., the addition of a reagent to the sample that is being observed, and the implementation of the desired change. The automated feedback system and methods described herein provide an automated system that includes computer-driven modifications to 1) allow for pre-determined experimental parameters to be achieved or maintained, 2) allow for real-time data analysis to conduce raw observational parameters to operatable parameters indicative of the status of the biological cells and their environment that are required for decision making, and 3) allow for remote monitoring, control and interaction with ongoing laboratory experiments in real-time by a laboratory researcher. This eliminates the need for the researcher to be present and controlling the flow of the experiment as it is being run. In addition, the systems and methods described herein provide a modular-based design that allows for the incorporation of previously disparate devices into an automated and integrated system through wired or wireless connectivity. A system according to the present disclosure may be referred to as a Lab Automation System.

In one aspect, the system utilizes digital microscope-reagent handing units (e.g., microfluidic pump units) and can be configured with any number of units added to the system, as desired. The web-based design of the systems and methods described herein allows researchers to remotely control the flow of and view the data collected by experiments and procedures that are being performed in a laboratory. Embodiments can be utilized for any experiment requiring visual measurement of material properties, and can be configured to respond to instructions to alter or view an experiment or procedure only with proper cryptographic identification for security purposes.

As disclosed herein, the automation system may be configured for use with fluorescence microscopy experiments and includes a computer which can perform image processing, and a fluid handling unit such as a microscope/microfluidic pump unit which is controlled by the computer. One or more applications executing on the computer handle image capture, image analysis, experiment control and provides a user interface for real-time interaction with the experiment, as described further herein below. In use, certain experimental parameters are set by the researcher, for example to maintain and achieve a certain illumination (or fluorophore concentration), the computer calculates aspirate and dispense regent volumes in order to maintain and/or achieve the illumination, converts the volume dosage to appropriate motor controls, and applies those controls to a motor that drives a syringe to administer or aspirate fluids or droplets in the laboratory. The experiment disclosed herein may be utilized to measure cellular response to various stimuli by measuring the fluorescence emitted by cells, and administering a variable feedback volume in response, or until measurement thresholds are reached. This allows a researcher who is at a remote location from the lab to study the intricate and continuous response of cells to various chemicals in order to investigate the effects of the chemicals and gain insight into disease to discover how cells function and communicate without being present in the laboratory to administer the feedback volumes necessary to reach the desired result.

In an aspect, a method for real-time assessment and monitoring of biological cells or their environment comprises

receiving a current readout of the biological cells or their environment;

assessing, by a computer, a change in the biological cells or their environment by comparing a current value of a parameter that describes the biological cells or their environment to a previous value of the parameter for the biological cells or their environment and/or a current or previous value for a comparative sample, wherein the current value of the parameter that describes the biological cells or their environment is determined from the current readout;

determining, by the computer, a treatment to be applied to the biological cells or their environment, the determining based on the assessing and on user specified criteria;

applying, by the computer, the treatment to the biological cells or their environment, the applying comprising controlling a setting on an environmental control unit to cause the treatment to be applied to the biological cells; and

optionally performing the assessing, determining, and applying on a periodic basis in response to receiving additional current readouts of the biological cells or their environment.

In one aspect, the method is a method for real-time assessment and monitoring of biological cells, the current readout is a current image of the biological cells, the current image including a microscope image acquired by a microscope; the treatment is an amount of reagent and the applying comprises controlling a setting on a reagent handling unit to cause the treatment to be applied to the biological cells.

As used herein, the term biological cells includes all types of biological cells, particularly those capable of being cultured, tissues containing biological cells, as well as entire organisms such as yeast, bacteria, viruses, and C. elegans and other multicellular organisms. Biological cells can be monitored for example by imaging using a microscope. Exemplary cells include cancer cells, neuronal cells, stem cells, immune cells, genetically modified cells, and other.

As used herein, the environment of biological cells includes a culture medium in the case of cultured cells, or an extracellular matrix in the case of tissues, or tissue engineered artificial environments. Extracellular signals in the media, environment or the extracellular matrix can be monitored and measured.

Readouts of biological cells or their environment include images, numbers, or a combination thereof. The open-source image analysis software such as Image J can be used for processing biological images. Alternatively, MatLab can be used for processing both images and numerical data. Custom-generated software can also be used (and potentially interfaced with ImageJ, MatLab, and/or some other analytical suite) to perform this functionality.

Exemplary readouts of the biological cells or their environment include microscopy images, luminescence readouts, fluorescence readouts, pH readouts, concentration readout of soluble factors including ion, growth factors, cytokines, neurotransmitters, amino acids, enzymes, and the like. The readouts of the cells can include detection of changes in the cells themselves, and/or changes in the environment of the cells including the concentration of analytes in the media or extracellular matrix. Analytes include cellular products produced by the cells and transported or excreted out of the cells, as well as components of the media, for example, that are internalized by the cells.

Exemplary devices which can be used to obtain a readout of the cells or their environment include microscopes, a biosensor array, a luminescence detector, an array of sensing electrodes, an array of pH meters, a thermistor, a spectrometer, and the like. For example, in the case of bioluminescence or bio-optic data, either luminescence or fluorescence signals can be detected. An array of electrodes can be used with high temporal resolution to measure ion influxes and electrochemical potentials, or an array of pH meters can be used to monitor the ionic properties of the environment of a cell across a two-dimensional expanse.

In one aspect, receiving a current readout of the biological cells or their environment comprises automated readout acquisition such as automated microscopic image acquisition. open source software (such as TWINE or Micro-Manager) can be used to capture and analyze images from a standard fluorescence microscope. Also, when the readout is a microscope image, automated shutter control can be employed to minimize excitation light exposure. Separate shutter control can be added as an accessory module and the device driver can be integrated into the central control software. In addition, automated microscope stage control can also be employed. Automated stage control can be employed to track a sample over hours, days or even weeks. The stage can be installed as an accessory module and its driver adapter integrated to the “central control software”.

Exemplary parameters that describe the biological cells and their environment include a size of the biological cells, a density of the biological cells, a membrane characteristic of the cells, characteristics of intracellular organelles including mitochondria, the Golgi apparatus, the endoplasmic reticulum, lysosomes, peroxisome, and synaptic vesicles, a cytoskeletal characteristic including microtubule, actin and other microfilaments. a cytoplasmic characteristic of the cells, a nucleus characteristic of the biological cells, a chromatin structure of the biological cells, a nucleolus characteristic of the biological cells, a morphology of the biological cells, an adhesion property of the cells, cell death within the biological cells, cell dormancy within the biological cells, cell proliferation, cell movement, cell specialization within the biological cells, characteristics of cell-cell junctions and inter-connectivity, characteristics of cellular products from specialized cellular processes including lipid vacuole, mucus, pigmentation, characteristics of cell pathological features including Nissel bodies, amyloid tangles and plaques, tau tangles, degenerative fragmentation of cellular structures, a concentration of an analyte external to the biological cells or a combination thereof.

In one aspect, the assessing by a computer, a change in the biological cells or their environment by comparing a current value of a parameter that describes the biological cells or their environment to a previous value of the parameter for the biological cells or their environment, for example, comprises accessing a previous readout of the biological cells or their environment and comparing the current readout of the biological cells or their environment to the previous readout of the biological cells or their environment. In one aspect, the current readout is taken at a specified time interval after the previous readout. The current readout can be taken after a perturbation to the biological cells or their environment, and the previous readout can be taken prior to the perturbation to the biological cells or their environment. When the biological cells are monitored by microscopy, the assessing comprises accessing a previous image of the biological cells and comparing the current image of the biological cells to the previous image of the biological cells. The current image can be taken after a perturbation to the biological cells, and the previous image can be taken prior to the perturbation to the biological cells.

In another aspect, assessing, by a computer, a change in the biological cells or their environment is by comparing a current value of a parameter that describes the biological cells or their environment to a current or previous value for a comparative sample such as a diagnostically related sample. The analysis by a computer essentially converts raw measurements to readouts that indicate the status of the biological cells and their environment. These readouts describe the fundamental characteristics of living organisms, therefore are comparable across samples, species, and the like, regardless of the mode of measurement or converting algorithms. Examples include, converting cell count of a unit area/volume to a value of cell density; converting morphological measurement of cell protrusions normalized to cell areas to values of a property of cell growth and/or cell adhesion; measurement of a certain ion concentration in a cell and convert it by normalizing to time intervals to generate a value to indicate ion flux, and calculation of cells with different morphology and convert to values of certain cell type percentage as values to indicate cell differentiation. It is thus diagnostically relevant to take data from diagnostically related samples, for example, (such as healthy controls or other samples from the same patient/tissue) into consideration during a decision-making process. Data points that examine overall growth of tumor cells across all of the plates from a single biopsy could give algorithmic weight to a new treatment approach/drug.

Exemplary perturbations of the biological cells or their environment include the addition of a drug, the addition of a labeling reagent, the addition of a cell-secreted factor, a change in media, a change in pH, a change in turbidity, a change in conditions, or a combination thereof. Changes in conditions include a change in humidity, temperature, light intensity, osmotic pressure, atmospheric conditions, media turbidity, physical factors that impact cell growth, or a combination thereof. For example, applying physical force to the samples or to deform them physically can provide valuable insight. These perturbations may or may not be applied in an effort to simulate a more accurate in vivo model.

The method also includes determining, by the computer, a treatment to be applied to the biological cells or their environment, the determining based on the assessing and on user specified criteria. Exemplary treatments include an amount of a reagent, a change in humidity, a change in temperature, a change in light intensity, a change in osmotic pressure, a change in atmospheric conditions, or a combination thereof. Exemplary reagents include drugs, labeling reagents, cell-secreted factors, media, pH-adjusting agents, ionic agents, particulate agents, and genetic materials such as transgene, microRNA, shRNA, viruses, plasmids, drugs, nanoparticles, and combinations thereof. For example, in one aspect, the assessment shows that the media has been depleted of essential factors, and the treatment comprises adding fresh media to the cell culture. In another aspect, a treatment is applied to the environment of the biological cells which mimics some aspect of in vivo environment of the sample being monitored.

When the treatment is the amount of a reagent, the method optionally comprises determining, by the computer, the type of the reagent.

For liquid delivery of an amount of a reagent, in certain embodiments, a minute volume (1-200 uL) of drug delivery, or a larger volume (200 uL-5 mL) liquid exchange may be employed. In the case of minute volume delivery, a syringe pump that is controlled by open source software Ardulno can be employed by the lab automation system. Larger volumes may be used for liquid media exchange for solution-based assays. Commercial bioreactor technologies, software-controlled valves and gravity-based liquid collection reservoirs may be employed.

One important feature of the methods described herein is that determining the treatment to be applied to the cells is determined by a combination of assessing the change (or a current state) in the cells or their environment by a computer and by the application of user specified criteria. For example, a user can set a positive cancer cell growth criteria to be 1) a cell colony area expands for >50% within 3 days, and 2) cell proliferation assay measurement to be >certain fluorescence signal intensity threshold over 3 days, and if the sample meets the criteria, a certain drug will be applied to the culture. The computer measures the two parameters, calculates the values for growth and proliferation, and compares the values to previous time point to determine the changes. If these values meet the criteria, the computer sets the trigger for action 1, applying a certain drug.

In an additional example, the treatment begins at one value but sweeps through a range of values/treatments as it hones in on what settings produce the most (positive, negative, etc.) change over time. For example, an experiment may include two cultures but they are set up as to always receive the exact same treatment: a healthy piece of tissue and a biopsied cancer tissue. They may or may not share the same physical media. In operation, the device could conceivably start with one chemotherapeutic dose, but lower the dose by 5% if it notices the healthy control reacting too poorly to continue, or increase the dose by 5% if the healthy tissue is unaffected and the tumor tissue is growing too rapidly. By sweeping through a range of values/drugs/regimens, the device can locate a highly personalized solution that affects the cancer cells without exceeding a maximum “harm to control cells” threshold, classified algorithmically as we've described elsewhere. This could even take the form of a “Rate of healthy deterioration vs Rate of cancerous deterioration” plot, with adjustable hysteresis.

The user-specified criteria allow the device to mimic the decision-making process of a knowledgeable professional, but allows the decision-making process to be done in real-time, allowing more accurate “biological system models” than those designed previously. Essentially, by allowing the computer to perform the assessment, determine the treatment and apply the treatment, the process can be achieved on a much faster time scale than could be accomplished by a laboratory worker.

In one aspect, the performing of the assessing a change, determining a treatment to be applied, and applying the treatment occur on a periodic basis in response to receiving additional current readouts of the biological cells or their environment.

According to an aspect, the receiving a current readout of the biological cells or their environment is in response to a request, by the computer, to a readout device to obtain the current readout of the biological cells or their environment. The current readout is received from the readout device. For example, the users may have access to a screen that displays all current data reads along with analysis, and that updates as quickly as everything is received. For example, the images taken can be more or less ‘movie-like’ depending on how frequently they were set to be taken. This data-capture frequency can be determined on an application-to-application basis by the scientist at the start of a protocol. Additionally, the rate of “applying” could have certain rules, such as “wait 5 minutes between drug applications,” which can also be applied by the user.

In one aspect, the requesting the current readout and the performing is automated. For example, an eventuality could be programmed wherein the system observes a specific stage or type of tumorous growth in an otherwise healthy tissue, then switches into a higher data-polling frequency to take more minute observations, or to display more/different information to the user. This could also give the machine a better chance at quickly classifying the observed phenomenon.

In certain aspects, the method further comprises outputting, via a user interface over a network, at least one of the current readout of the biological cells or their environment, the change in the biological cells or their environment, the current value of the parameter, the previous value of the parameter, the user specified criteria, the treatment, and the setting on the environmental control unit.

In other aspects, the method further comprises receiving, via a user interface over a network, a request to modify at least one of the user specified criteria, the treatment, and the setting on the environmental control unit; and modifying the at least one of the user specified criteria, the treatment, and the setting on the environmental control unit in response to receiving the request.

Thus, when using an embodiment of the lab automation system, a professor can log onto the system (e.g., through a host computer and/or network) and pull up any relevant data from any sample/experiment/etc. that the professor has authority to access. The professor can also selectively intervene at any point during the process, with potential restrictions being implemented depending no clinical/experimental setting.

In certain aspects, the user specified criteria includes at least one of a threshold for the parameter and a weighting value of the parameter. Exemplary thresholds for change of the parameters include the magnitude and or time-dependence of the parameters.

The change in the biological cells can be classified as one of a positive change, no change, and a negative change. The change in the biological cells is used to determine the treatment to be applied to the cells, such as a routine that maintains temperature, or one that lyses the cells.

In another aspect, the method further comprises collecting an extracellular product that has been produced by the biological cells. For example, one could study the extracellular (or even intracellular) product by collection, monitoring, speed of accumulation, or total product observed.

In one aspect, the treatment to be applied to the cells is determined using a trigger-based routine system. In a trigger-based routine system, the applying is performed immediately after the determining, based on pre-programmed parameter/treatment routines and without user intervention.

In one example, a given sample goes through stages in early development where it first creates a given number of stem cells, then immediately begins differentiation once a very specific condition is met. Exemplary conditions include an extracellular concentration of protein X, a physical stimulus Y, or a confluency of Z. In order to simulate or model such an environment, one would want a device to constantly monitor the sample and be prepared for triggers X, Y, and/or Z to reach the threshold values and to administer an accurate and real-time response.

In another example, a given producer-species begins producing after a certain stage of development. Depending on the size of the culture, a user may collect extracellular samples once an extracellular yield of X is achieved, performing this once at a low value of X, then at a higher value, and so forth, until a maximum yield where the cells should be immediately lysed and the yield collected. The ability to take the hypothesized directives immediately once these values are reached is an advantage of the system and methods disclosed herein.

In an aspect, the method is for real-time assessment and monitoring of biological cells, the current readout is a current image of the biological cells, the current image including a microscope image acquired by a microscope; the treatment is an amount of reagent and the applying comprises controlling a setting on a reagent handling unit to cause the treatment to be applied to the biological cells. In a more specific aspect, the biological cells are tumor cells and the perturbation of the biological cells is the addition of a first cancer drug. If it is determined, for example, that the addition of the first cancer drug does not achieve the desired outcome in the cells, the reagent can be a second cancer drug, or an increased or decreased amount of the first cancer drug.

The methods and devices described herein allow for the administration of drugs based on feedbacks from machine-acquired readouts from patient-derived cell and tissue culture systems in order to address the inherent difficulties of personalized medicine approaches such as large sample variance. With the advent of patient-derived cells, it is currently possible to generate personalized tissue culture as patient-specific “test bed” for individually tailored drug development. Patient-derived tumor tissues can be cultured in vitro in conditions that closely mimic the tumor microenvironment in the body. These systems provide more realistic cancer models for drug screening. However, the heterogeneity inherent in individualized testing introduces technical barriers to high-throughput drug screening as sample-variant parameters need to be determined.

Conventional drug-screening is carried out in static culture systems of cells and tissues using immortalized cell lines. The approach of in vitro cell-based drug screening has been successful in identifying general drug candidates, it remains restrictive for selecting drugs targeting primary tumors that are composed of heterogeneous cell populations. Batch testing prohibits sample-variant drug dosing and timing. Moreover, parameters are often determined in a short-term setting and are therefore unlikely to uncover long-term effects. Lastly, although more options are available for treating a particular disease, there is increasing difficulty in deciding which drug is most appropriate for an individual patient. In reality, a cancer patient may need to go through several rounds of drug treatment after the first-line of chemotherapy fails; however, selection for new treatment regimen is extremely challenging given the vast heterogeneity of tumor profiles and individual differences. There are no protocols to guide decision-making for sequential drug treatment at present. Clearly, there is a mismatch between the conventional practices of batch-based drug screening with the real-world experience of an individual patient.

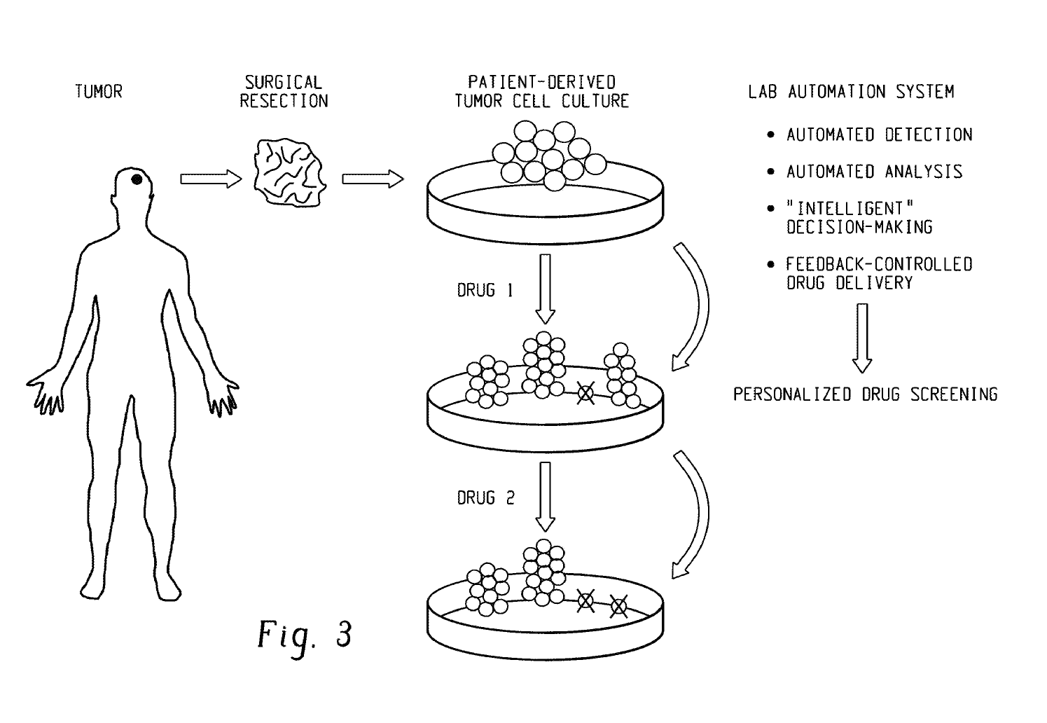

The systems and methods described herein will advance drug development for personalized medicine (FIG. 3). As a patient's diseased tissue is cultured outside the body, the systems and methods described herein provide real-time, automated monitoring of cellular activities and drug responses. Importantly, with automated data analysis and algorithm-based “intelligent” decision-making, the system provides feedback-controlled drug delivery and initiate iterative drug screening. This system provides a platform for tailored design of drug treatment for individual patient's tissue. Furthermore, the biological samples with “unexpected” drug responses can be detected in real time to allow for tissue harvest for more detailed profiling of the molecular signatures for identifying new drug candidates; and the new information can be built into the algorithm of the feedback-based control. The system and methods also allow for monitoring of the same sample for hours, days or weeks as the sample may be stored away from the system and then re-placed on the system at a later time to allow for a current readout to be taken.

By combining digital microscopy and solution-based analysis, the systems and methods described herein provide multi-mode assessment of phenotypic and target-based effects of cancer drugs on patient-derived cell cultures. These readouts are analyzed in real-time with an algorithm that incorporates prior knowledge of the user to control drug administration controlled by a computer.

Advantageously, in the systems and methods described herein, a computer is used to automatically “change” the “Potentially simulated” environment of the cell cultures based on altered biological signals. In contrast to a population analysis used for batch-based drug screening, data can be gathered from the same sample before and after perturbations. By tracking cellular responses in real time ad over long periods of time, the consistency of environmental variables is maintained.

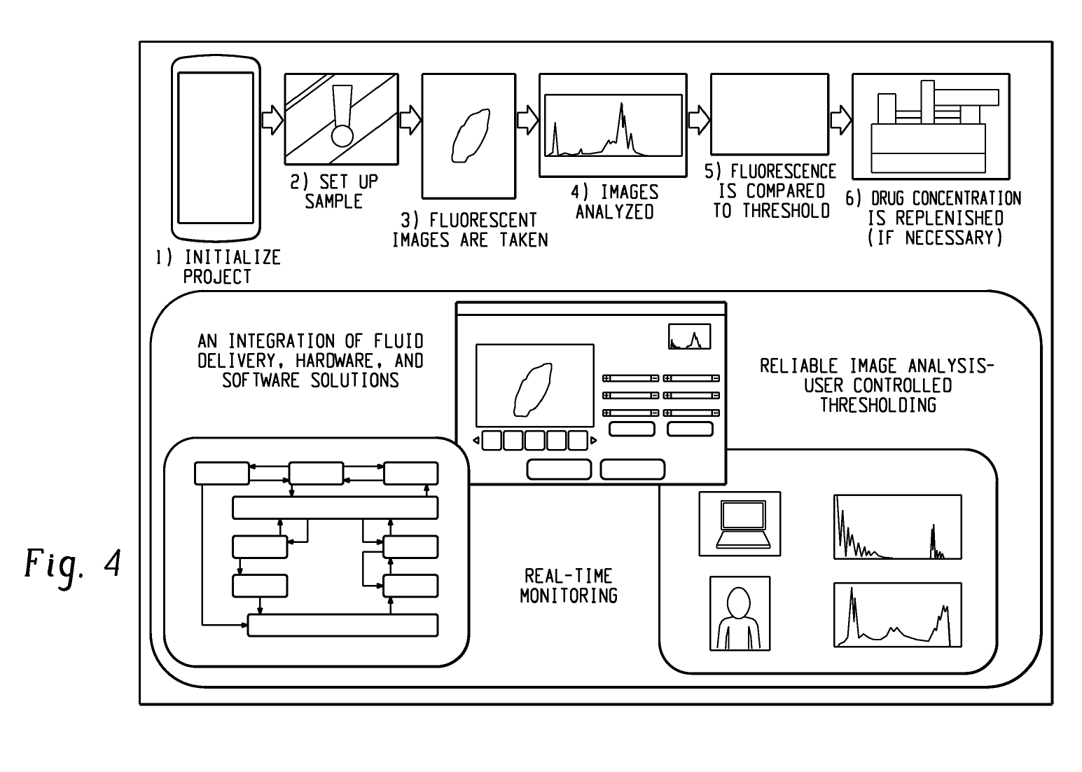

FIG. 4 shows a prototype of a system according to the present disclosure. The prototype automatically captures microscopy images of living cells, and based on pre-set criteria defined by the user, such as an increase of fluorescence signal due to activated biological activity, triggers a microfluidic pump that administers a drug into the cell culture in order to dampen the activity. The system calculates signal changes via automated image analysis, such as a histogram as shown in FIG. 4, and determines the extent of the pump action, such as drug dosing and timing. This automated process allows sensitive detection of low-level signal changes of the cellular activities in real-time, such as calcium signaling in a neuron (shown in FIG. 4), as well as accurate drug administration in minute levels (1-10 μL). Importantly, the feedback loop between cell responses and drug dispensing is performed automatically based on pre-set criteria (i.e., the intensity peak value in the histogram), therefore allowing feedback-based control of the fluid pump action. In addition, the feedback control allows for a much faster response to the change in biological activity than could be achieved by a human user.

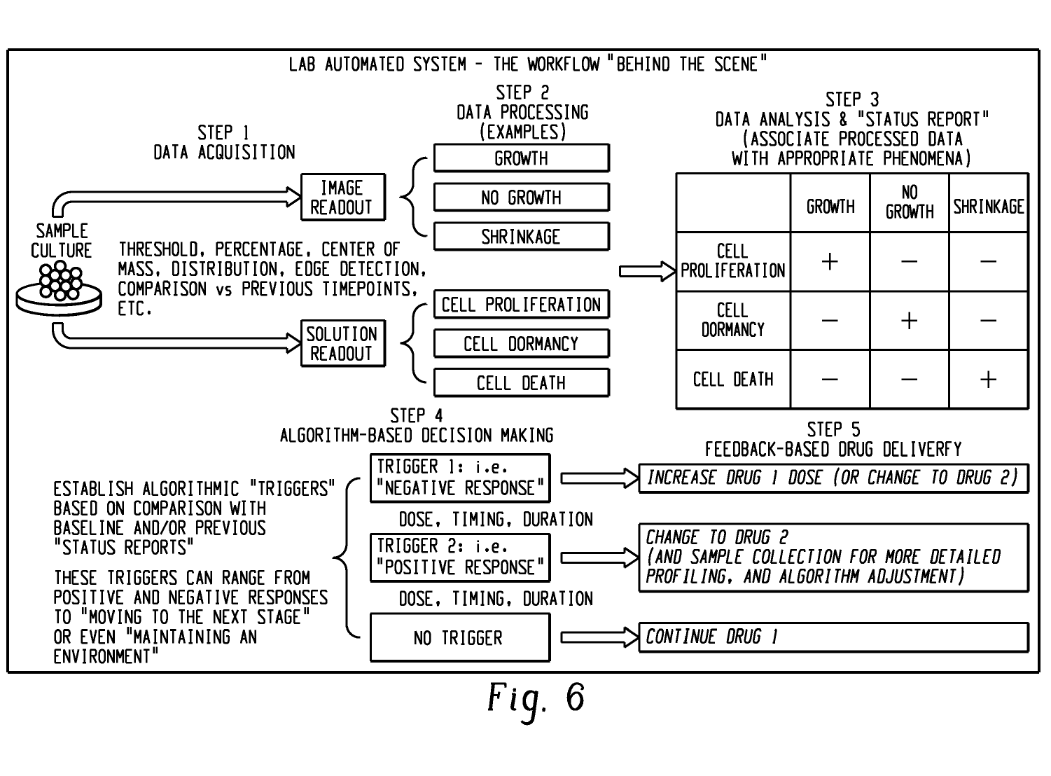

In one aspect, the system includes a standard cell/tissue culture vessel such as a single well or multi-well plate. Alternatively, the system can include a chamber with multiple connectors and/or liquid exchange capabilities. In one aspect, an enclosed chamber with a microscopy-compatible glass bottom hosts build-in connection ports to inlet and outlet tubing that connect with peripheral devices (e.g., a readout device for obtaining a current readout of the biological cells, and a environmental control unit for applying a treatment to the biological cells) with standard adaptors. This design allows for easy setup at the time of experiment. However, the flow path may need to be primed to ensure bubble-free and leak-proof liquid flow. In a second aspect, a custom-designed lid that can fit with standard cell culture vessel, such as a petri dish, has built-in connection ports that can be pre-assembled with the peripheral devices. In this design, it is important to keep the imaging area free of optical obstruction on the microscope stage. FIG. 6 is a schematic of an experimental chamber.

In one aspect, a system for real-time assessment and monitoring of biological cells or their environment, comprising

a means for interfacing to a microscope,

a cell chamber for obtaining microscopic images of the biological cells or their environment using the microscope, the cell chamber comprising an inlet and an outlet, wherein the inlet and the outlet allow for flow of a reagent through the cell chamber,

a reagent handling unit in fluid communication with the inlet of the cell chamber for dispensing reagent through the inlet of the cell chamber, and

a computer configured for accepting the microscopic images from the microscope, for processing the microscopic images from the microscope, and for controlling the reagent handling unit based on the processing.

The means for interfacing to a microscope can include, but is not limited to GUI with icons representing appropriate commands and a current display from the microscope, a wide display where regions can be targeted individually and stage movement is handled automatically to focus on these regions, typed input, an API for some other environment (such as MATLAB or the an OS command prompt/terminal) involving commands that more directly interface with the software that controls the device, or a combination thereof.

The microscope is an example of a readout device and the reagent handling unit is an example of an environmental control unit.

In one aspect, the system further comprises an automated shutter control unit for the microscope. In another aspect, the system further comprises an automated stage control unit for the microscope. In yet another aspect, the system further comprises a spectrometer for determining a spectrometric readout of a liquid sample from the environment of the biological cells.

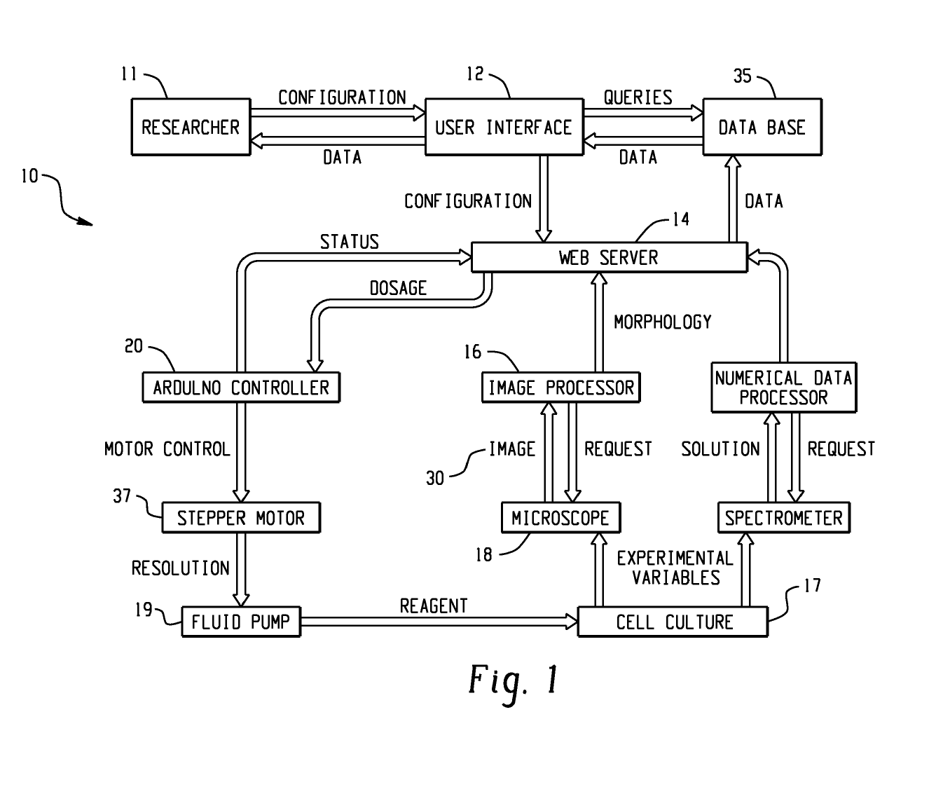

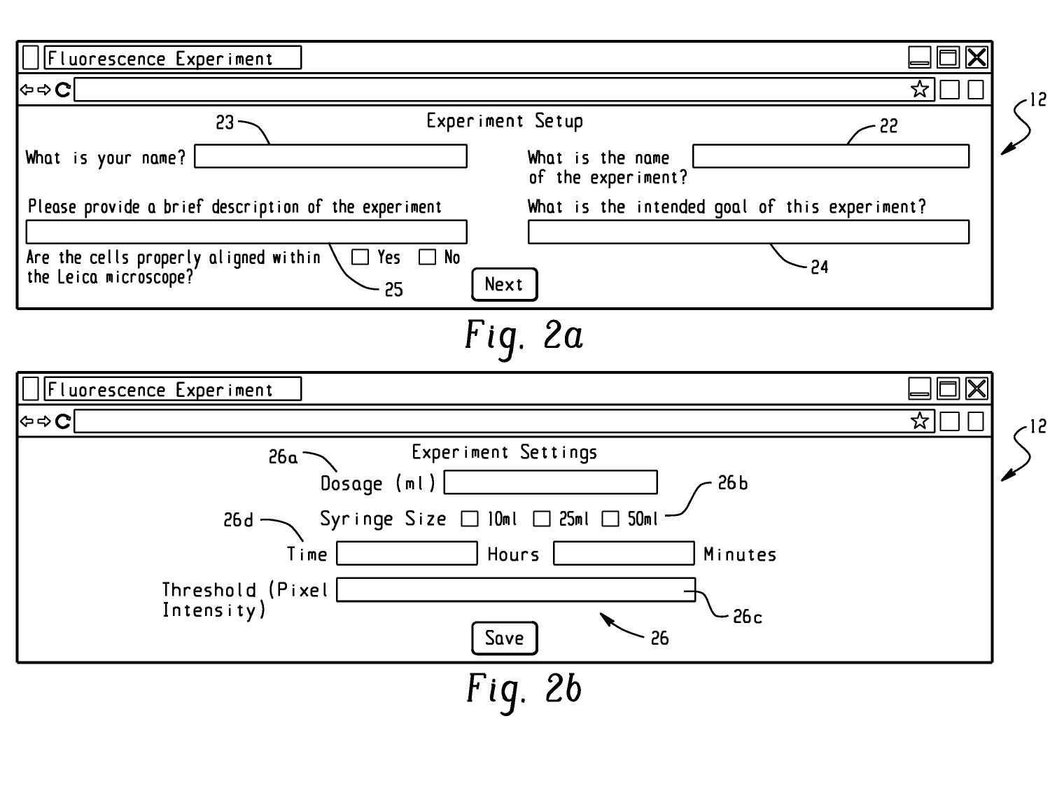

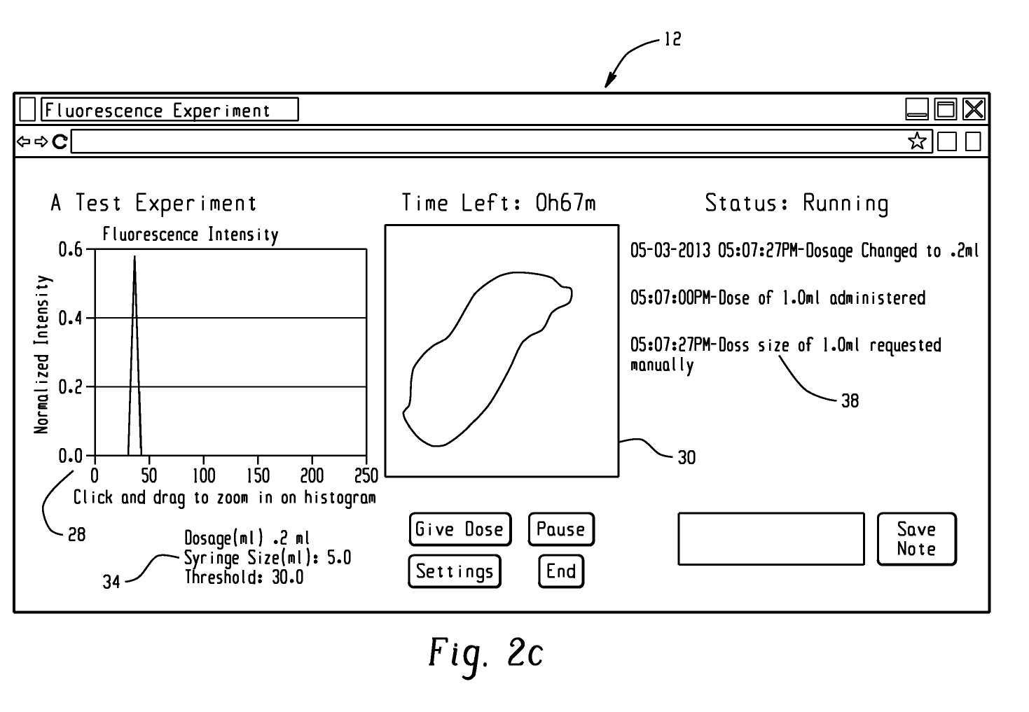

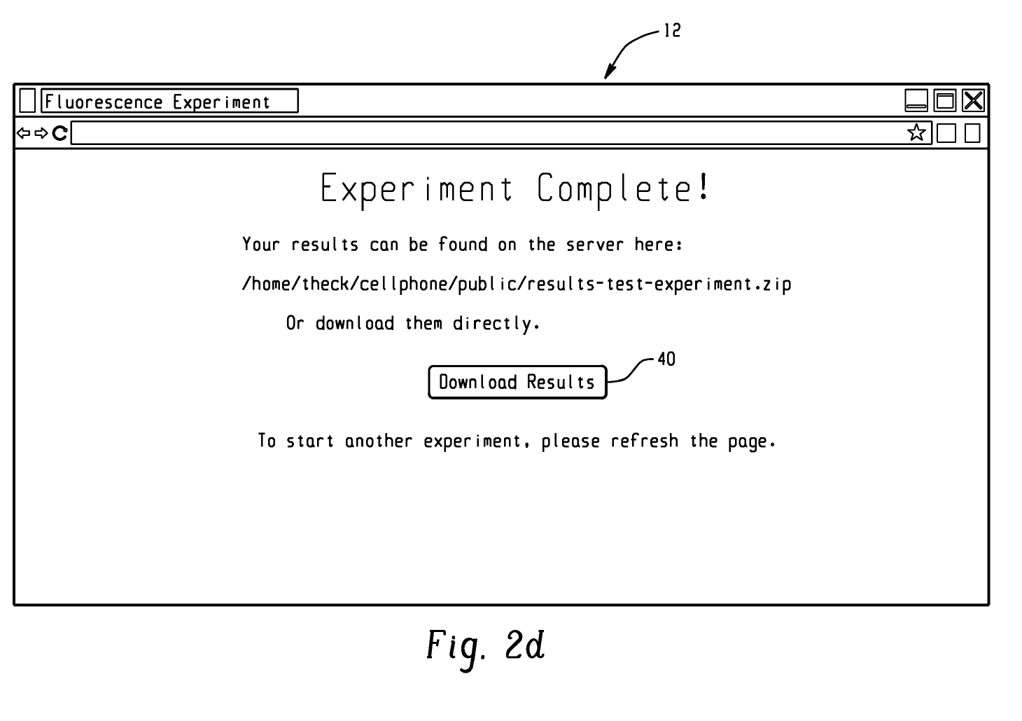

Referring initially to FIGS. 1 and 2a-2d, an exemplary embodiment of a laboratory automation system 10 includes a user interface 12, at least one web server 14, an image processor 16, a microscope 18, a microfluidic pump 19 unit and an Ethernet operated microcontroller 20. The laboratory automation feedback system 10 shown in FIG. 1 allows for remote monitoring, control and interaction with ongoing laboratory experiments in real-time by a researcher, which eliminates the need for the researcher to be physically at the lab during the running of the experiment. The system 10 is also a “smart” or “intelligent” system, which is programmed with pre-defined goals or parameters, for example maintaining a certain illumination of the cells (which can be mathematically equated to a certain drug or fluorophore concentration) in the present embodiment. The system 10 employs analysis-based decision-making, which is based upon reactions noted during experimentation, and analyzes qualitative, and often complicated data by utilizing processing and analysis techniques that would otherwise have to be performed manually by the laboratory researcher. This allows the system 10 to dynamically generate and/or alter experimental commands (for example aspirate/dispense, etc.) mid-protocol without a researcher's input.

The user interface 12 acts as the interface between the researcher and a potential array of experiments, provides an easily navigable gateway to set up an experiment's configuration and parameters; to monitor, control and access data for the experiment, and is accessible from any computer or mobile device connected to the local network that runs the web server. For example, the user interface 12 can be used to input and edit experimental configurations for both clerical and experiment details, including such fields as experiment title 22 and expected results 24, as well as settings for current experiment parameters 26. The user interface 12 also may provide controls that allow the user to log notes on the state of the experiment, to prematurely end the experiment, to alter dosage settings during the experiment, to manually administer drugs, and to otherwise edit experimental settings in real-time.

As shown in FIG. 2a, initially the user interface 12 is utilized by a researcher to set up the parameters for the experiment according to the particular experiment. In the present embodiment, the parameters may include, for example, a description of the experiment 25, dosage amount 26a, syringe size 26b, threshold (i.e. pixel intensity) 26c and experiment time 26d. The user interface 12 is served by at least one web server 14, which may be composed of, but is not limited to HTML Abstraction Markup Language (HAML), JavaScript, cascading style sheets (CSS), and some Java Script Object Notation (JSON) data. HAML is an HTML derivative that can contain embedded Ruby code, and is eventually parsed into pure HTML when sent to the user. In the present embodiment, the HAML and CSS can provide the structure and content of the webpage as viewed by the researcher and the JavaScript sent to the browser can be used to display complex graphical elements and dynamically update pages as the experiment progresses, as described herein below. An external JavaScript library, such as highcharts.js, can be used to display image histogram data in the present embodiment as also described herein below. Customized JavaScript can be used to send asynchronous JavaScript and XML (AJAX) requests to the web server, presenting the researcher with the most current experimental data through the user interface 12.

Once the experiment parameters are set up through the user interface 12, the web server 14 can coordinate data flow for the experiment. In the present embodiment, at least one web server 14 may include one or more web servers, which may be implemented as Sinatra applications. Sinatra is a minimal Ruby framework for web applications that allows for easy use of the Hypertext Transfer Protocol (HTTP) in a multithreaded environment. If more than one web server is utilized, the primary web server may run two main processes, one responding to HTTP requests and serving web content, and the other performing experiment tasks such as image capture and dosage automation, as described herein below. The secondary, or image processing web server may be responsible for responding to requests to generate the histogram data of an image saved to a database and with JSON data.

At least one web server 14 coordinates dataflow by requesting images from the microscope 18, forwarding the requested images to an image processor 16 for processing (for example a histogram server), and can send both the image histogram 28 and the original image 30 to the laboratory technician for review through the user interface 12 (FIG. 2c). The microscope 18 may be any conventional light microscope that includes an onboard camera, which captures magnified images of the cell culture utilizing various filters (selecting for various spectral ranges) applied to capture different types of fluorescence, as desired. Photoluminescent data is received from sample fluorescing cells on the microscope stage, as would be known to those of skill in the art. For the image capture functionality, the TWAINCommander command line program, can be used in the present embodiment.

The web server 14 can send image capture requests directly to the microscope 18 and also receives fluorescent digital images from the digital microscope 18. The web server 14 also sends the digital images received from the microscope 18 to the image processor, in this embodiment the Histogram server 16a, to be analyzed. The image processor 16 develops an image captured from the microscope 18 and measures luminescence levels found in the image. In the present embodiment, this can be achieved through simple pixel intensity analysis, but the system can be equipped for any form of signal processing as may be required and as would be known to those of skill in the art. After the image processor 16 receives fluorescent captures from the web server 14 and analyzes them, the image processor 16 sends fluorescence levels to the web server 14 where they can be viewed through the user interface 12. In the present embodiment, the web server 14 receives intensity histograms from the histogram server 16a that are accessible to the researcher through the user interface 12 (FIG. 2c). The original digital images 30 are also accessible through the user interface 12, and may be saved in a database 35 of the web server 14 in JPEG or other format system, where they remain accessible through the user interface 12.

The web server 14 can further orchestrate the experiment automation dataflow by comparing the threshold injection value 26c to the current average intensity threshold 34 of the experiment and manage dosage administration via the microcontroller 20 and syringe pump 19, an Arduino microcontroller being utilized with the syringe pump 19 in the present embodiment. The system 10 evaluates whether and what type of dosage should be administered based upon the pre-defined goals or parameters originally set by the researcher and the data received. A stepper motor 37 can be provided that drives the syringe pump 19 to administer or aspirate fluids or droplets in the laboratory. The web server 14 can continuously receive confirmation pings from the microcontroller 20 for dosage instructions, which are calculated by the system to determine aspirate and dispense volumes in order to maintain and/or achieve the particular experimental parameters, such as cell illumination in the present embodiment. The system then translates the dosage instructions to aspirate/dispense requests to control signals for the micro-fluid pump 19. This involves conversion of dosage requested to the proper amount of rotational motion for the stepper motor 37, and compensation for various syringe sizes. In the present embodiment for the experiment described, the Arduino-controlled pump can be programmed using compatible C libraries along with a JSON library. The stepper motor 37 shown in FIG. 1 includes a threaded drive shaft, and can be powered by the Arduino microcontroller 20. By utilizing the unique incremental nature of the stepper motor, the Arduino is able to rotate the shaft a precise number of degrees, which corresponds to a linearly related lateral distance. By attaching an Ethernet shield, the Arduino can also be connected to the webserver 14 and query for dosage instructions, delivered in JSON. Experimental data and settings may also be received from the technician via the web user interface 12 in addition to manual dosage requests.

The web server 14 sends dosage requests and experimental settings to the Arduino-controlled pump 19 which outputs administered doses as directed. If measurement thresholds are reached, the lab technician can also administer a feedback volume remotely. In the present embodiment, JSON objects containing information on dosage volumes and syringe size from the web server are sent over Ethernet or wireless network. Buttons that allow the user to manually adjust the placement of the drive shaft may also be provided. The output of doses of chemicals may be made in the range of tenths of milliliters, although it is not so limited. In addition to the foregoing, it should be understood that this control can be expanded to additional types of pumps and controllers.

Use of the laboratory automation system 10 shown in FIG. 1 will now be described with respect to a fluorescence experiment which measures cellular response to stimuli by measuring the fluorescence emitted by the cells and with reference to the Figures. A researcher 11 logs on to the user interface 12, for example by providing a user name and password, or other cryptographic identification for security purposes. The researcher 11 then sets up the experiment by entering, through the user interface 12, the fields for the experiment, such as the experiment title 22, description of the experiment 25, expected results 24, and the parameters 26 for the experiment and the name of the researcher 23. For the present experimental example, the parameters 26 may include the dosage amount 26a, syringe size 26b, threshold (i.e. pixel intensity to achieve a certain illumination or fluorophore concentration) 26c and experiment time 26d. The researcher 11 also prepares the laboratory equipment, for example the microscope 18 and syringe 19 by plating fluorescently tagged cells 17 on slides and preparing the stimuli, as would be known to one of skill in the art. The researcher 11 can now leave the laboratory and allow the at least one web server 14 to coordinate data flow for both the image processing and managing dosage administration as described herein.

The at least one web server 14 coordinates image processing by requesting images from the microscope 18, forwarding the requested images to the Histogram server 16a for processing and sends both the image histogram 28 and the original image 30 to the user interface 12 for evaluation by the researcher 11. Likewise, the at least one web server 14 manages dosage administration by sending dosage requests and experimental settings to the Arduino-controlled pump 19 which outputs administered doses as directed. The web server 14 continuously receives confirmation pings from the Arduino-microcontroller 20 for dosage instructions, and the web server 14 compares the threshold injection value 26c to the current average intensity threshold 32 of the experiment to intelligently manage dosage administration 34 by first determining if a dosage is required, and if a dosage is required determining the amount of the dosage, and administering the dosage via the microcontroller 20 and micro-fluid pump 19. If measurement thresholds have not been reached, the web server 14 indicates this to the microcontroller 20, which translates aspirate/dispense requests to control signals for the micro-fluid pump 19. This involves conversion of dosage requested to the proper amount of rotational motion for the stepper motor 37, and determines compensation for various syringe sizes. If measurement thresholds are reached the system may determine not to administer a dose. In either case, a researcher 11 can administer a feedback volume manually, remotely and in real-time through the user interface 12. During the running of the experiment the researcher 11 can check on the experiment's status 38 through the user interface 12 and intervene remotely and in real-time to change the parameters of the experiment, for example by changing dosages. After sufficient data has been collected, the fluorescence of the cells deteriorates, or the cells' exposure to environmental conditions reaches a maximum, the researcher 11 ends the experiment and the accumulated data can be downloaded 40 in the database 35 for further analysis.

The invention is further illustrated by the following non-limiting examples.

### Example 1: Pilot Study of Cancer Drug Screening

FIG. 6 illustrates the workflow for cancer drug screening. Drugs used to target malignant brain tumors will be tested on human tumor samples. From image analysis, readouts such as growth” (bigger size), “no growth” (no size change), “shrinkage” (smaller size) can be determined. Solution readouts include cell proliferation, cell dormancy and cell death based on specific assay outcomes, i.e., above-threshold spectrometric reading. These data feed into an algorithm that generates a multiplexed matrix as a “status report” of the cell culture. A status report at a specific time point is compared with those at previous time points to determine whether the drug that the cells are exposed to is “ineffective” (i.e., tissue growth and cell proliferation), or “resistance” (i.e., no growth and cell dormancy), or “drug working” (i.e., tissue shrinkage and cell death). The algorithm-based decision-making will activate the liquid handling devices and deliver drugs accordingly, either changing existing drug doses or drug types, or continue the current drug regimen. In order to use this system to guide drug screening effort, the system will alert the researchers to take cell samples at a specific state, for example, when the cells are found to be resistant to standard chemotherapy drugs. This sample can then be used for more in-depth proteomic and genomic profiling to identify potential new drug candidates.

More specifically, a test run will be performed on brain tumor cell lines, such as U87, a human primary glioblastoma cell line. This cell line, is widely used as malignant brain tumor model for in vitro drug screening. Their growth characteristics are well documented, so the parameters of growth rate and phenotypic features can be found in the literature which can be used to generate default settings in the Lab Automation System. Cultured U87 cells will be used for long-term culture (2 wk-3 mon), and with a daily imaging session (approximately lhr/per session) on the Lab Automation System, generate the image readout. Live cell assays for proliferation, cell cycle and cell death (apoptosis) will be used to generate the solution-based readout.

To test drugs, standard chemotherapeutic agents (Table 1) to generate “status reports” consisting of cell responses to each drug in long-term culture (2 wk-3 mon) for U87 cell culture. The “status reports” will be compared with previously published findings and used to validate or optimize the system settings. The validated data will be used to provide a reference for primary tumor cell cultures. The expectation is that the system will detect and respond to phenotypic and activity changes of tumor cell lines under drug treatment, and produce results that are consistent with literature reports.

TABLE 1
Brain tumor and standard Drug Treatments

| Brain tumor Types | Standard chemotherapy agents |
|---|---|
| Glioblastoma multiforme | Temodar (oral), avastin (IV) |
| | carmustine (IV), irinotecan (IV) |
| Medullablastoma | Lomustine + vincristine + |
| | cisplatin (IV combination), retinoic acid |
| | or carboplatin (rescue therapy) |
| Ependymoma | Ciaplatin (IV), etoposide (IV), |
| | carboplatin (IV), vincristine (IV) |

Next, long-term cultures will be generated from primary brain tumors that are surgically removed from pediatric patients. These cultures will be tested for standard chemotherapeutic agents (Table 1) based on their clinical diagnosis. The default settings from U87 cell culture will be used as a starting point. Standard 96-well plate cultures will be performed as controls to carry out manual drug screening as described by established batch-based drug screening protocols. The Lab Automation System will be compared with manual testing, including responsiveness of drugs, cellular activities, and labor input, reagent usage, etc. The system will detect and respond to drug responses of patient-derived tumor cell cultures, and produce meaningful results.

### Example 2: Exemplary Laboratory Automation System

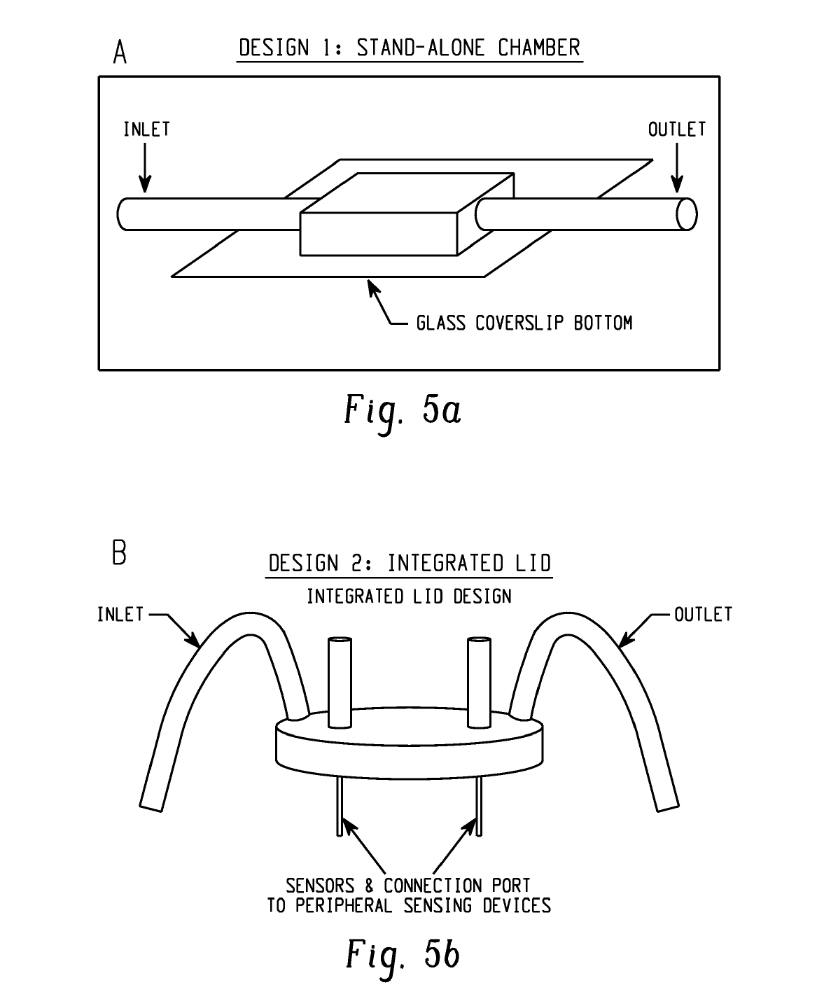

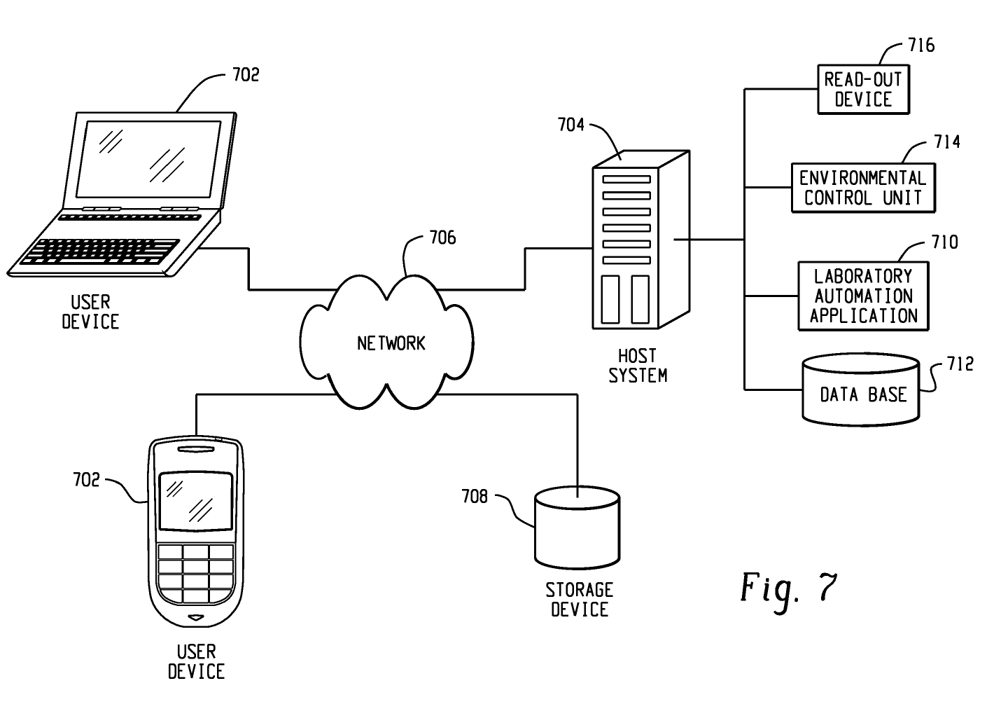

Turning now to FIG. 7, a block diagram of an exemplary laboratory automation system is generally shown in accordance with an embodiment. The system includes a laboratory automation application 710 that is executed by one or more computer programs located on a host system 704 to perform the processing described herein. The system depicted in FIG. 7 also includes one or more user systems 902 through which users (e.g., scientists, lab technicians, professors, etc.) at one or more geographic locations may contact the host system 704. In an embodiment, users perform the processing described above in FIGS. 2-6 via user interfaces on the user systems 702. The user systems 702 can be coupled to the host system 704 via a network 706. Each user device 702 may be implemented using a general-purpose computer executing a computer program for carrying out the processes described herein. The user systems 702 may be personal computers (e.g., a lap top, a tablet computer, a cellular telephone) or host attached terminals. If the user systems 702 are personal computers, the processing described herein may be shared by a user device 702 and the host system 704.

The network 706 may be any type of known network including, but not limited to, a wide area network (WAN), a local area network (LAN), a global network (e.g. Internet), a virtual private network (VPN), and an intranet. The network 706 may be implemented using a wireless network or any kind of network implementation known in the art. A user device 702 may be coupled to the host system through multiple networks (e.g., cellular and Internet) so that not all user systems 702 are coupled to the host system 704 through the same network. One or more of the user systems 702 and the host system 704 may be connected to the network 706 in a wireless fashion. In one embodiment, the network is the Internet and one or more user systems 702 execute a user interface application (e.g. a web browser) to contact the host system 704 through the network 706. In another exemplary embodiment, the user device 702 is connected directly (i.e., not through the network 706) to the host system 704. In a further embodiment, the host system 704 is connected directly to or contains the storage device 708. In an embodiment, the user systems 702 have support for user interface screens displayed on display devices that can be used for data input and/or output.

The storage device 708 includes data relating to the laboratory automation system and may be implemented using a variety of devices for storing electronic information. Though shown as a separate from the storage device 708, the database 712 may be completely or partially contained in the storage device 708. The term storage device 708 when used herein includes the data stored in the database 712. It is understood that the storage device 708 may be implemented using memory contained in the host system 704 or that it may be a separate physical device. The storage device 708 is logically addressable as a consolidated data source across a distributed environment that includes the network 706. Information stored in the storage device 708 may be retrieved and manipulated via the host system 704 and/or via a user device 702. The database 712 may be implemented using any technology that supports the processing described herein. For example, the database 712 may be implemented as a relational database with structured query language (SQL) queries used to access the data.

The host system 704 depicted in FIG. 7 may be implemented using one or more servers operating in response to a computer program stored in a storage medium accessible by the server. The host system 704 may operate as a network server (e.g., a web server) to communicate with the user device 702. The host system 704 handles sending and receiving information to and from the user device 702 and can perform associated tasks. The host system 704 may also include a firewall to prevent unauthorized access to the host system 704 and enforce any limitations on authorized access. For instance, an administrator may have access to the entire system and have authority to modify portions of the system. A firewall may be implemented using conventional hardware and/or software as is known in the art.

The host system 704 may also operate as an application server. The host system 704 executes one or more computer programs, including the laboratory automation application 710, to perform the functions described herein. Processing may be shared by the user device 702 and the host system 704 by providing an application to the user device 702. Alternatively, the user device 702 can include a stand-alone software application for performing a portion or all of the processing described herein. As previously described, it is understood that separate servers may be utilized to implement the network server functions and the application server functions. Alternatively, the network server, the firewall, and the application server may be implemented by a single server executing computer programs to perform the requisite functions.

Also shown in FIG. 7 is an environmental control unit 714 and a readout device 716 which are directly connected to the host system 704. In other embodiments, one or both of the environmental control unit 714 and the readout device 716 are connected to the host system 704 via the network 706. In addition, although shown as two separate devices, the environmental control unit 714 and the readout device 716 can be implemented as a single integrated device. Further, more than one environmental control unit 714 and/or the readout device 716 can be connected to the host system 704. The environmental control unit 714 can be implemented by any device that can apply a treatment to biological cells or their environment such as but not limited to: a microfluidic pump unit, a device for generating various wavelengths of light, an environmental controller responsible for heating/cooling, an environmental controller responsible for gas levels, an actuator for applying pneumatic pressure in sealed containers, a vacuum pump for maintaining a vacuums, electrodes and signal generators for applying electrostatic (or electromagnetic) fields, a physical perturbation unit such as a shaker or force applicator. The readout device 716 can be implemented by any device that can take a current readout of the biological cells or their environment, such as but not limited to: a microscope, spectroscope, luminometer, electrode array, patch clamp, pH meter, thermistor array, and the like.

The system shown in FIG. 1 is a particular embodiment of the system shown in FIG. 7. In an embodiment, the researcher 11 shown in FIG. 1 uses a user device 702 to access the laboratory automation application 710 to perform the processing described above. The user interface 12, web server 14, and image processor 16 shown in FIG. 1 can be implemented by the laboratory automation application 710 executing on the host system 704 shown in FIG. 7. Further, the Arduino controller 20, stepper motor 37 and fluid pump 19 shown in FIG. 1 together are an example implementation of the environmental control unit 714 shown in FIG. 7. Still further, the microscope 18 shown in FIG. 1 is an example implementation of the readout device shown 716 shown in FIG. 7. In addition, the database 35 shown in FIG. 1 can be implemented by one or both of the storage device 708 and database 712 shown in FIG. 7.

In one aspect, a system for real-time assessment and monitoring of biological cells or their environment, comprising

a memory having computer readable instructions; and

an environmental control unit, and one or more processors for executing the computer readable instructions, the computer readable instructions comprising:

receiving a current readout of the biological cells or their environment;

assessing a change in the biological cells or their environment by comparing a current value of a parameter that describes the biological cells or their environment to a previous value of the parameter for the biological cells or their environment and/or a current or previous value for a comparative sample, wherein the current value of the parameter that describes the biological cells or their environment is determined from the current readout;

determining a treatment to be applied to the biological cells or their environment, the determining based on the assessing and on user specified criteria;

applying the treatment to the biological cells or their environment, the applying comprising controlling a setting on the environmental control unit to cause the treatment to be applied to the biological cells; and

optionally performing the assessing, determining, and applying on a periodic basis in response to receiving additional current readouts of the biological cells or their environment.

In one aspect, a computer program product for real-time assessment and monitoring of biological cells or their environment comprising

a computer readable storage medium having program instructions embodied therewith, the program instructions executable by a processor to cause the processor to:

receive a current readout of the biological cells or their environment;

assess a change in the biological cells or their environment by comparing a current value of a parameter that describes the biological cells or their environment to a previous value of the parameter for the biological cells or their environment and/or a current or previous value for a comparative sample, wherein the current value of the parameter that describes the biological cells or their environment is determined from the current readout;

determine a treatment to be applied to the biological cells or their environment, the determining based on the assessing and on user specified criteria;

apply the treatment to the biological cells or their environment, the applying comprising controlling a setting on the environmental control unit to cause the treatment to be applied to the biological cells; and

perform the assessing, determining, and applying on a periodic basis in response to receiving additional current readouts of the biological cells or their environment.

Embodiments may utilize a cloud based delivery of the laboratory automation application 710 to provide a highly secure, fully resilient system. The use of cloud based delivery allows for shared infrastructure and support which may reduce system complexity and cost, as well as simplify maintenance and upgrades.

Embodiments may provide browser based access and full mobility support. An embodiment includes thin client access with support for all major browsers. Embodiments support tablet computers and handheld computers (e.g., cell phones).

Embodiments may provide interfaces to existing computer systems to retrieve or provide data. This allows for the laboratory automation system to operate along with existing systems. Embodiments support the secure exchange of data between systems in accordance with established security protocols and governmental regulations.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method for real-time assessment and monitoring of biological cells or their environment, the method comprising:
applying a perturbation to the biological cells or their environment,
receiving a current readout of the biological cells or their environment in response to the perturbation;
assessing, by a computer, a change in the biological cells or their environment by comparing a current value of a parameter that describes the biological cells or their environment to a previous value of the parameter for the biological cells or their environment taken prior to the perturbation and/or a current or previous value for a comparative sample, wherein the current value of the parameter that describes the biological cells or their environment is determined from the current readout;
determining, by the computer, a treatment to be applied to the biological cells or their environment, the determining based on the assessing and on user specified criteria;
applying, by the computer, the treatment to the biological cells or their environment, the applying comprising controlling a setting on an environmental control unit to cause the treatment to be applied to the biological cells or their environment;
optionally performing the assessing, determining, and applying on a periodic basis in response to receiving additional current readouts of the biological cells or their environment.

2. The method of claim 1, further comprising requesting, a readout device to obtain the current readout of the biological cells or their environment, wherein the receiving is from the readout device in response to the requesting, wherein the requesting and performing are automated.

3. (canceled)

4. The method of claim 1, wherein the current readout is a microscopy image, a luminescence readout, a spectrometer readout, a fluorescence readout, a pH readout, a concentration readout of a soluble factor, or a combination thereof.

5. The method of claim 1, wherein the parameter that describes the biological cells or their environment is a size of the biological cells, a density of the biological cells, a membrane characteristic of the biological cells, a characteristics of intracellular organelles of the biological cells, a cytoskeletal characteristic of the biological cells. a cytoplasmic characteristic of the biological cells, a nucleus characteristic of the biological cells, a chromatin structure of the biological cells, a nucleolus characteristic of the biological cells, a morphology of the biological cells, an adhesion property of the biological cells, cell death within the biological cells, cell dormancy within the biological cells, biological cell proliferation, biological cell movement, biological cell specialization, characteristics of cell-cell junctions, cell-cell inter-connectivity, characteristics of cellular products from specialized biological cellular processes, characteristics of biological cell pathological features, degenerative fragmentation of cellular structures, a concentration of an analyte external to the biological cells, or a combination thereof.

6. The method of claim 1, wherein the assessing comprises accessing a previous readout of the biological cells or their environment and comparing the current readout of the biological cells or their environment to the previous readout of the biological cells or their environment.

7. (canceled)

8. The method of claim 1, wherein the perturbation of the biological cells or their environment is the addition of a drug, the addition of a labeling reagent, the addition of a cell-secreted factor, a change in media, a change in pH, a change in turbidity, a change in conditions, or a combination thereof.

9. The method of claim 8, wherein the change in conditions is a change in humidity, temperature, light intensity, osmotic pressure, atmospheric conditions, or a combination thereof.

10. The method of claim 1, wherein the treatment to be applied to the biological cells or their environment is an amount of a reagent, a change in humidity, a change in temperature, a change in light intensity, a change in osmotic pressure, a change in atmospheric conditions, or a combination thereof.

11. The method of claim 10, wherein the reagent is a drug, a labeling reagent, a cell-secreted factor, media, a pH-adjusting agent, an ionic agents, a particulate agent, or a combination thereof.

12. The method of claim 1, wherein the method is for real-time assessment and monitoring of biological cells or their environment, the current readout is a current image of the biological cells, the current image including a microscope image acquired by a microscope; the treatment is an amount of reagent and the applying comprises controlling a setting on a reagent handling unit to cause the treatment to be applied to the biological cells or their environment.

13. The method of claim 12, wherein the biological cells are tumor cells and a perturbation of the biological cells is the addition of a first cancer drug.

14. The method of claim 13, wherein the reagent is a second cancer drug.

15. The method of claim 13, wherein the reagent is an increased or decreased concentration of the first cancer drug.

16. The method of claim 12, further comprising determining, by the computer, the type of the reagent.

17. The method of claim 1, further comprising outputting, via a user interface over a network, at least one of the current readout of the biological cells or their environment, the change in the biological cells or their environment, the current value of the parameter, the previous value of the parameter, the user specified criteria, the treatment, and the setting on the environmental control unit.

18. The method of claim 1, further comprising:
receiving, via a user interface over a network, a request to modify at least one of the user specified criteria, the treatment, and the setting on the environmental control unit; and
modifying the at least one of the user specified criteria, the treatment, and the setting on the environmental control unit in response to receiving the request.

19. The method of claim 18, wherein the user specified criteria includes at least one of a threshold for the parameter and a weighting value of the parameter.

20. The method of claim 1, wherein the change in the biological cells is classified as one of a positive change, no change, and a negative change.

21. The method of claim 1, wherein the applying is performed immediately after the determining, based on pre-programmed routines and without user intervention.

22. A system for real-time assessment and monitoring of biological cells or their environment, comprising
a means for interfacing to a microscope,
a cell chamber for obtaining microscopic images of the biological cells or their environment using the microscope, the cell chamber comprising an inlet and an outlet, wherein the inlet and the outlet allow for flow of a reagent through the cell chamber,
a reagent handling unit in fluid communication with the inlet of the cell chamber for dispensing reagent through the inlet of the cell chamber, and
a computer configured for accepting the microscopic images from the microscope, for processing the microscopic images from the microscope, and for controlling the reagent handling unit based on the processing, wherein controlling the reagent handling unit comprises determining the amount and type of reagent to add to the biological cells or their environment.

23. The system of claim 22, further comprising an automated shutter control unit for the microscope.

24. The system of claim 22, further comprising an automated stage control unit for the microscope.

25. The system of claim 22, further comprising a spectrometer for determining a spectrometric readout of a liquid sample from the environment of the biological cells.

26. A system for real-time assessment and monitoring of biological cells or their environment, the system comprising:
a memory having computer readable instructions; and
an environmental control unit, and one or more processors for executing the computer readable instructions, the computer readable instructions comprising:
applying a perturbation to the biological cells or their environment;
receiving a current readout of the biological cells or their environment in response to the perturbation;
assessing a change in the biological cells or their environment by comparing a current value of a parameter that describes the biological cells or their environment to a previous value of the parameter for the biological cells or their environment taken prior to the perturbation and/or a current or previous value for a comparative sample, wherein the current value of the parameter that describes the biological cells or their environment is determined from the current readout;
determining a treatment to be applied to the biological cells or their environment, the determining based on the assessing and on user specified criteria;
applying the treatment to the biological cells or their environment, the applying comprising controlling a setting on the environmental control unit to cause the treatment to be applied to the biological cells or their environment; and
optionally performing the assessing, determining, and applying on a periodic basis in response to receiving additional current readouts of the biological cells or their environment.

27. A computer program product for real-time assessment and monitoring of biological cells or their environment, the computer program product comprising a computer readable storage medium having program instructions embodied therewith, the program instructions executable by a processor to cause the processor to:
apply a perturbation to the biological cells or their environment;
receive a current readout of the biological cells or their environment in response to the perturbation;
assess a change in the biological cells or their environment by comparing a current value of a parameter that describes the biological cells or their environment to a previous value of the parameter for the biological cells or their environment taken prior to the perturbation and/or a current or previous value for a comparative sample, wherein the current value of the parameter that describes the biological cells or their environment is determined from the current readout;
determine a treatment to be applied to the biological cells or their environment, the determining based on the assessing and on user specified criteria;
apply the treatment to the biological cells or their environment, the applying comprising controlling a setting on the environmental control unit to cause the treatment to be applied to the biological cells or their environment; and
optionally perform the assessing, determining, and applying on a periodic basis in response to receiving additional current readouts of the biological cells or their environment.

28. (canceled)
